(19) United States
(12) Patent Application Publication
(10) Pub. No.: US 2023/0310735 A1
(43) Pub. Date: Oct. 5, 2023

(54) ELECTRONICALLY CONTROLLED INTRAVENOUS INFUSION PUMP

(71) Applicant: ICU Medical, Inc., San Clemente, CA (US)

(72) Inventors: Robert P. Cousineau, Boston, MA (US); Ivo P. Toptchev, San Diego, CA (US); Mark C. Rohlwing, Mesa, AZ (US); James R. Shults, Ramona, CA (US); Yei Feng Moy, Buffalo Grove, IL (US); Eric Kutchery, Paddock Lake, WI (US)

(21) Appl. No.: 18/190,878

(22) Filed: Mar. 27, 2023

Related U.S. Application Data
(60) Provisional application No. 63/325,060, filed on Mar. 29, 2022.

(51) Int. Cl. A61M 5/142; A61M 39/22

(52) U.S. Cl. CPC A61M 5/142; A61M 39/22

(57) ABSTRACT

Disclosed are embodiments of electronic intravenous infusion pumps. In some embodiments, a medical infusion pump system can include a plurality of processors that are operationally isolated from each other. Some pump systems can include a modular electromechanical pump driver comprising a plurality of components that can be removed as a unit from within the pump housing without removing the components from each other. Some pump systems can include a handle formed as a unitary structure, the handle not including any portion of the boundary between panels of the pump housing. Some pump systems can include a memory within the pump housing that is configured to contain information about one or more pumping parameters for execution by at least two of the pump drivers. Some pump systems can include a connector configured to separately receive and secure each one of a plurality of different-sized IV pole stands. Some pump systems can include one or more accelerometers configured to indicate whether the pump system has fallen. Some pump systems can include one or more temperature sensors configured to modify the performance of one or more operations of the pump system in response to an increase in temperature above an acceptable operating temperature.

### RELATED APPLICATIONS

This application claims the benefit under 35 U.S.C. § 119(e) of U.S. Provisional Patent Application No. 63/325,060, filed on Mar. 29, 2022, and entitled, “ELECTRONICALLY CONTROLLED INTRAVENOUS INFUSION PUMP,” the entire content of which is hereby incorporated by reference herein and made a part of this specification for all that it discloses.

### BACKGROUND

### Field

This disclosure relates to intravenous infusion pumps, including electronically controlled intravenous infusion pumps.

### Related Art

Patients all over the world who are in need of medical care commonly receive intravenous infusion therapy, especially during surgery or when hospitalized. This generally involves inserting a needle into a patient's blood vessel, usually in the hand or arm, and then coupling the needle to a catheter in communication with one or more different types of therapeutic fluids. Once connected, the fluid travels from the fluid source(s), through the catheter, and into the patient. The fluid can provide certain desired benefits to the patient, such as maintaining hydration or nourishment, diminishing infection, reducing pain, lowering the risk of blood clots, maintaining blood pressure, providing chemotherapy, and/or delivering any other needed drug or other therapeutic liquid to the patient. Electronic infusion pumps in communication with the fluid sources and the patient can help to increase the accuracy and consistency of fluid delivery to patients, but current electronic infusion pumps have disadvantages.

### SUMMARY

In some embodiments, a medical infusion pump system can include: a pump housing; an electromechanical pump driver positioned within the housing, the pump driver configured to receive a disposable fluid holder and to pump medical fluid through the fluid holder; an electronic pump motor controller positioned within the housing, the pump motor controller being in electrical communication with the pump driver; and one or more additional electronic controllers within the housing, the one or more additional electronic controllers comprising one or more of a user interface controller in electrical communication with a user interface, or a communications engine in electrical communication with a communicator; wherein the electronic pump motor controller is operationally isolated from the one or more additional controllers in that the electronic pump motor controller and the one or more additional controllers are each configured to be capable of continuing operation when the other has stopped working, entered a failure mode, rebooted, or reset.

In some embodiments, a medical infusion pump system can include: a pump housing; a user display; a modular electromechanical pump driver positioned within the pump housing, the modular electromechanical pump driver comprising a first component in the form of a motor, a second component in the form of a loader configured to receive a disposable fluid holder, and a third component in the form of a plunger configured to pump medical fluid through the fluid holder; wherein the first, second, and third components of the modular electromechanical pump driver are directly or indirectly connected to each other as a removable unit, and wherein the modular electromechanical pump driver can be removed as a unit from within the pump housing without removing the first, second, and third components from each other.

In some embodiments, a medical infusion pump system can include: a pump housing formed of a plurality of discrete panels with one or more boundaries between the panels of the pump housing; a user display; an electromechanical pump driver positioned on or within the pump housing; a battery positioned on or within the pump housing; and a handle connected to the pump housing, the handle formed as a unitary structure and the handle not including any portion of the boundary between panels of the pump housing.

In some embodiments, a medical infusion pump system can include: a pump housing; a plurality of electromechanical pump drivers at least partially contained within the pump housing; a battery positioned on or within the pump housing; and one or more electronic processors within the pump housing in electrical communication with the plurality of pump drivers; and a memory within the pump housing that is associated with the one or more electronic processors, the memory configured to contain information about one or more pumping parameters executed by or to be programmed for execution by at least two of the pump drivers.

In some embodiments, a medical infusion pump system can include: a pump housing; a display; a processor; at least one electromechanical pump driver at least partially contained within the pump housing; a power source on or within the pump housing; and a connector configured to separately receive and secure each one of a plurality of different-sized IV pole stands, the connector comprising a first surface that is perpendicular to a second surface, each of the first surface and the second surface being configured when the connector receives and secures each one of the plurality of different-sized IV pole stands to simultaneously contact said pole stand.

In some embodiments, a medical infusion pump system can include: a pump housing; a display; at least one processor; at least one electromechanical pump driver at least partially contained within the pump housing; a power source on or within the pump housing; and one or more accelerometers configured to produce one or more electronic signals that indicates whether the pump system has fallen.

In some embodiments, a medical infusion pump system can include: a pump housing; a display; at least one processor; at least one electromechanical pump driver at least partially contained within the pump housing; a power source on or within the pump housing; and one or more temperature sensors in electronic communication with the processor; wherein the processor is configured to modify the performance of one or more operations of the pump system in response to an increase in temperature above an acceptable operating temperature.

### BRIEF DESCRIPTION OF THE DRAWINGS

The following drawings and the associated descriptions are provided to illustrate embodiments of the present disclosure and do not limit the scope of the claims.

FIGS. 1A-E show front perspective, front elevational, rear elevational, top plan, and side elevational views, respectively, of an example of an infusion pump.

FIG. 2 shows an example of a cassette that can be used with the pump of FIG. 1.

FIG. 3 shows a schematic diagram of functional electronic components for an example of a medical pump system.

FIGS. 4A-4B show side perspective views of the infusion pump illustrating removal, servicing, and/or replacement of a pump driver.

FIG. 5 is an exploded perspective view of the pump driver of FIG. 4B.

FIG. 6 is a rear perspective view of a portion of the housing of the pump of FIG. 1A that includes an exploded view of a handle assembly.

FIG. 7 is a top rear-sectional view of the pump of FIG. 1.

### DETAILED DESCRIPTION

This specification provides textual descriptions and illustrations of many devices, components, assemblies, and subassemblies. Any structure, material, function, method, or step that is described and/or illustrated in one example can be used by itself or with or instead of any structure, material, function, method, or step that is described and/or illustrated in another example or used in this field. The text and drawings merely provide examples and should not be interpreted as limiting or exclusive. No feature disclosed in this application is considered critical or indispensable. The relative sizes and proportions of the components illustrated in the drawings form part of the supporting disclosure of this specification, but should not be considered to limit any claim unless recited in such claim.

### Examples of Pump Systems

In some embodiments, a pump system can include a reusable pump driver and a disposable fluid holder, such as a fluid cassette, syringe, section of tubing, etc. A disposable cassette, which is typically adapted to be used only for a single patient and/or only for one fluid delivery cycle, is usually a small plastic unit having at least one inlet and an outlet respectively connected through flexible tubing to the fluid supply container and to the patient receiving the fluid. In some embodiments, the cassette can include a pumping chamber. The flow of fluid through the chamber can be controlled by a plunger or pumping element activated in a controlled manner by the reusable pump driver. For example, the cassette chamber can have one wall formed by a flexible diaphragm against which the plunger is repeatedly pressed in a reciprocating manner, which causes the fluid to flow. The pump driver can include the plunger or pumping element for controlling the flow of fluid into and out of the pumping chamber in the cassette, and it may also include one or more controls and/or vents to help deliver the fluid to the patient at a pre-set rate, in a pre-determined manner, for a particular pre-selected time, and/or at a pre-selected total dosage.

In some embodiments, the fluid can enter a cassette through an inlet and can be forced through an outlet under pressure. The fluid is delivered to the outlet when the pump plunger forces the membrane into the pumping chamber to displace the fluid. During the intake stroke, the pump plunger draws back, the membrane covering the pumping chamber retracts or pulls back from its prior inwardly displaced position, and the fluid is then drawn through the open inlet and into the pumping chamber. In a pumping stroke, the pump plunger forces the membrane back into the pumping chamber to force the fluid contained therein through the outlet. By repeating this action in an electronically controlled manner, the fluid flows into and out of the cassette in a series of spaced-apart pulses rather than in a continuous flow. When the pulses occur in rapid succession, the flow approximates a continuous flow. The entire disclosure of U.S. Pat. No. 7,258,534 is incorporated by reference herein, for all purposes, for all that it contains, including but not limited to examples of pump drivers and disposable fluid holders. It is contemplated that any structure, material, function, method, or step that is described and/or illustrated in the '534 patent can be used with or instead of any structure, material, function, method, or step that is described and/or illustrated in the text or drawings of this specification.

### Examples of Pump System Components

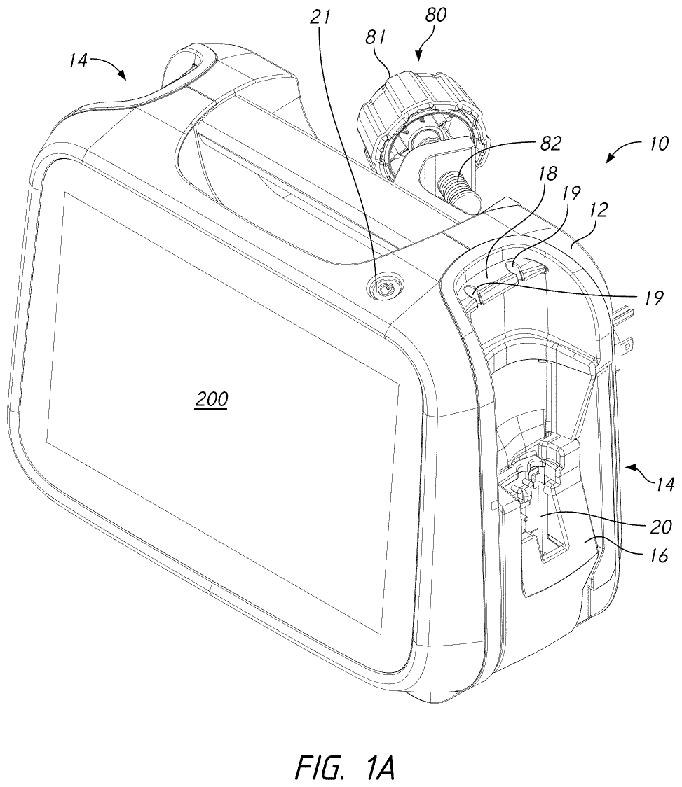

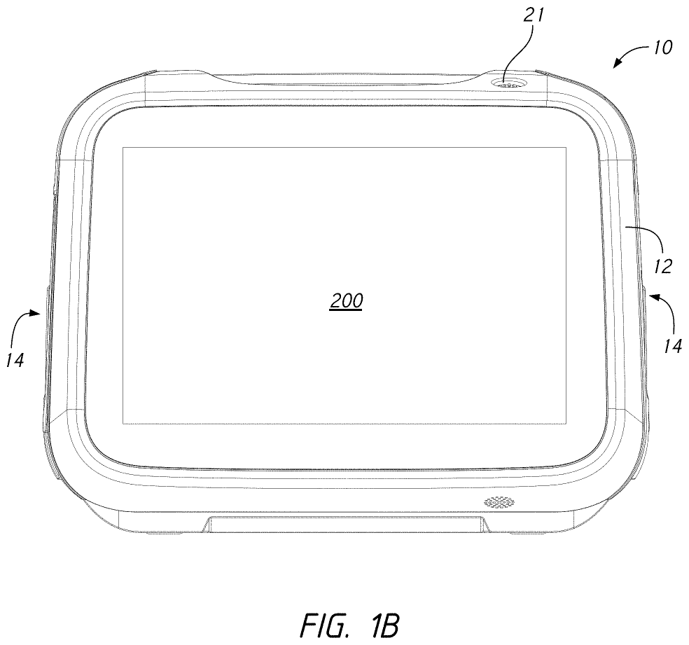

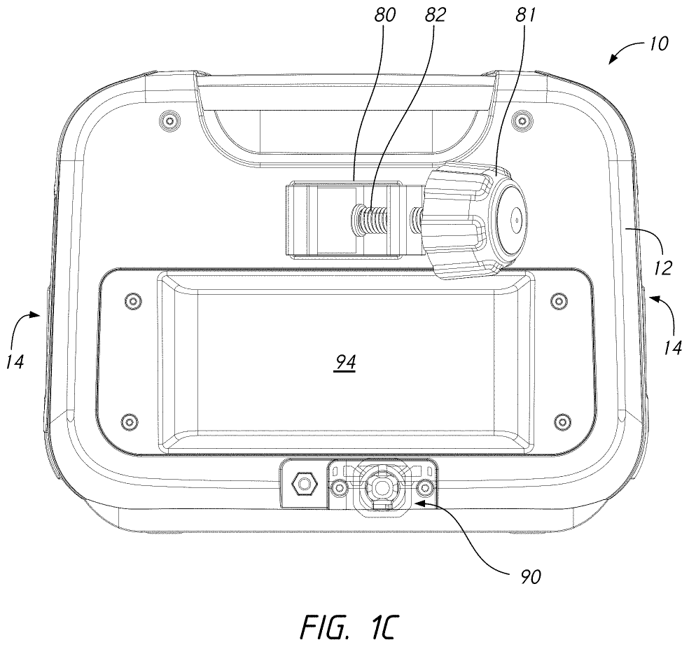

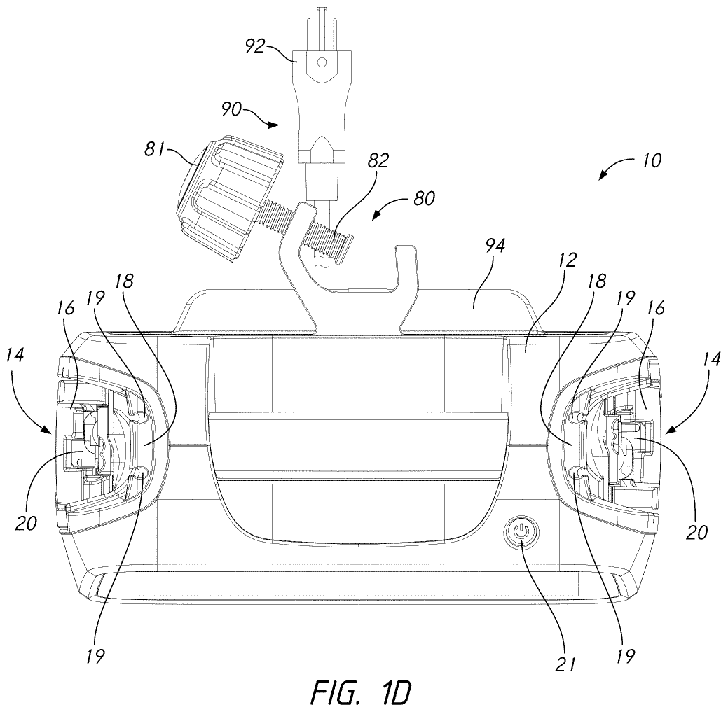

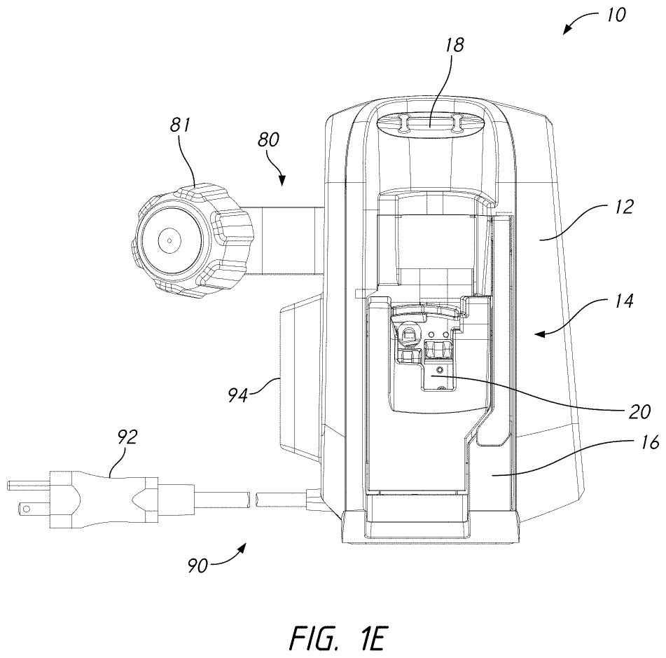

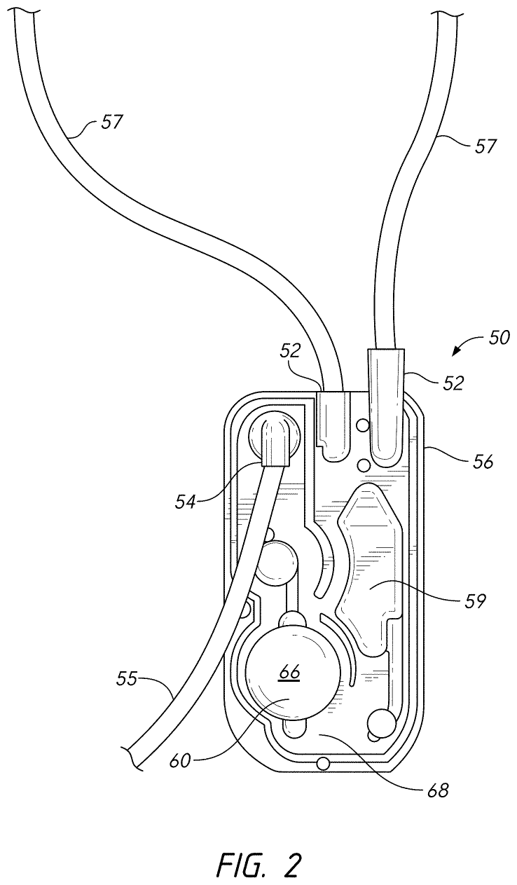

FIGS. 1A-1E show an electronic medical intravenous pump 10 with a housing 12 and at least one pump driver 14 attached to the housing 12. As illustrated, a plurality of pump drivers 14 (e.g., at least two) can be integrally provided, contained, and/or partially contained, within the same housing 12 of a single medical pump 10. Either or both of the pump drivers 14 can include a cover 16 that partially or entirely encloses an outer surface of the pump driver 14, an indicator 18 (e.g., an illuminating communicator) attached to the cover 16, one or more tube holders 19, and a loader 20 configured to securely receive and releasable hold a disposable fluid holder (see, e.g., FIG. 2), including but not limited to a cassette, syringe, and/or tubing. The one or more tube holders 19 can be configured to removably receive and securely hold one or more fluid-conveying tubes extending into or exiting from fluid holder when the fluid holder is received into the loader 20. The indicator 18 can communicate one or more messages to a user, such as by temporarily illuminating in one or more colors. Examples of one or more message include confirming that a pump driver 14 near the indicator is currently active and pumping or that one or more instructions being received from a user will apply to a pump driver 14 near the indicator 18. The loader 20 can be a mechanism with multiple moving parts that opens, closes, expands, contracts, clasps, grasps, releases, and/or couples with the fluid holder to securely hold the fluid holder on or within the pump 10 during fluid pumping into the patient. The loader 20 can be integrated into and positioned on or within the pump 10 near the cover 16 adjacent to the indicator 18.

A user communicator, such as display/input device 200, can be provided to convey information to and/or receive information from a user (e.g., in an interactive manner). As illustrated, the user communicator is a touch screen that is configured to provide information to a user through an illuminated dynamic display and is configured to sense a user's touch to make selections and/or to allow the user to input instructions or data. For example, the display-input device 200 can permit the user to input and see confirmation of the infusion rate, the volume of fluid to be infused (VTBI), the type of drug being infused, the name of the patient, and/or any other useful information. The display-input device 200 can be configured to display one or more pumping parameters on a continuing basis, such as the name of the drug being infused, the infusion rate, the volume that has been infused and/or the volume remaining to be infused, and/or the elapsed time of infusion and/or the time remaining for the programmed course of infusion, etc. As shown, the touch screen can be very large, for example at least about 4 inches x at least about 6 inches, or at least about 6 inches x at least about 8 inches. In the illustrated example, the touch screen fills substantially the entire front surface of the pump 10 (see FIG. 1A), with only a small protective boundary surrounding the touch screen on the front surface. As shown, the touch screen comprises at least about 80% or at least about 90% of the surface area of the front of the pump 10. In some implementations, the front of the touch screen comprises a clear glass or plastic plate that can be attached to the housing 20 in a manner that resists liquid ingress, such as using a water-proof gasket and/or adhesive that can withstand repeated exposure to cleaning and sanitizing agents commonly used in hospitals without significant degradation.

An actuator 21 can be provided separate from the user communicator. The actuator 21 can be configured to receive an input and/or display information to a user. As shown, the actuator 21 is a power button that permits the user to press on the actuator 21 to power up the pump 10. The actuator 21 can illuminated to communicate to the user that the pump 10 is powered on, such as by illuminating in green. If the power source is running low, the actuator 21 can change the color of illumination to quickly show to a user that a power source needs to be replenished, such as by illuminating in yellow. When the power source is nearly exhausted, the actuator can change the color of illumination, such as by illuminating in red.

In some embodiments, the user communicator, such as a display/input device 200, can alternatively or additionally comprise one or more screens, speakers 502, lights, haptic vibrators 504, electronic numerical and/or alphabetic read-outs, keyboards, physical or virtual buttons, capacitive touch sensors, microphones, and/or cameras, etc.

During use, the pump 10 is typically positioned near the patient who is receiving fluid infusion from the pump 10, usually lying in a bed or sitting in a chair. In some embodiments, the pump 10 may be configured to be an ambulatory pump, which will typically include a smaller housing, user communicator, battery, etc., so as to be conveniently transportable on or near a mobile patient. In many implementations, the pump 10 is attached to an IV pole stand (not shown) adjacent to the patient's bed or chair. As shown, the pump 10 can include a connector 80 that is configured to removably attach the pump 10 to the IV pole stand. As shown, the connector 80 can comprise an adjustable clamp with a large, easily graspable user actuator, such as a rotatable knob 81, that can be configured to selectively advance or retract a threaded shaft 82. At an end of the shaft 82 opposite from the knob 81 is a pole-contacting surface that can be rotatably advanced by the user to exert a force against a selected region of the pole, tightly pushing the pole against a rear surface of the pump 10, thereby securely holding the pump 10 in place on the pole during use. The selected region of the pole where the contacting surface of the shaft 82 is coupled can be chosen so as to position the pump 10 at a desired height for convenient and effective pumping and interaction with the patient and user.

The pump 10 can include a power source 90. In some embodiments, the power source can comprise one or more channels for selectively supplying power to the pump 10. For example, as illustrated, the power source 90 can comprise an electrical cable 92 configured to be attached to an electrical outlet and/or a portable, rechargeable battery 94. One or more components of the pump 10 can operate using either or both sources of electrical power. The electrical cable 92 can be configured to supply electrical power to the pump 10 and/or supply electrical power to the battery 94 to recharge or to maintain electrical power in the battery 94.

Inside of the housing 20 of the pump 10, various electrical systems can be provided within the housing to control and regulate the pumping of medical fluid by the pump 10 into the patient and/or to communicate with the user and/or one or more other entities. For example, the pump 10 can include a circuit board with a processor that includes a user interface controller (UIC) that is in electronic communication with and is configured to control and interact with a user interface, such as a graphical user interface, that can be displayed on the user communicator or display/input device 200. The pump 10 can include a printed circuit board with a processor that includes a pump motor controller (PMC) that is in electronic communication with and is configured to control one or more pump drivers 14. In some embodiments, the PMC is located on a separate circuit board from the UIC and/or the PMC is independent from and separately operable from the UIC, each of the PMC and UIC including different electronic processors capable of concurrent and independent operation. In some embodiments, there are at least two PMC's provided, a separate and independent one for each pump driver 14, capable of concurrent and independent operation from each other.

The pump 10 can include a printed circuit board with a processor that includes an electronic controller that comprises a communications engine (CE) in electrical communication with a communicator that enables and controls electronic communications between the pump 10 and other entities (aside from the user), such as electronic, wired or wireless, communication with a separate or remote: user, computer, computer system, server, hospital electronic medical records system, remote healthcare provider, router, network, another pump, a mobile electronic device, a near field communication (NFC) device such as a radio-frequency identification (RFID) device, and/or a central computer controlling and/or monitoring multiple pumps 10, etc. The CE can include or can be in electronic communication with an electronic transmitter, receiver, and/or transceiver capable of transmitting and/or receiving electronic information by wire or wirelessly (e.g., by Wi-Fi, Bluetooth, cellular signal, etc.).

In some embodiments, the CE, PMC(s), and/or the UIC are each located on a separate circuit board and in a different location within the housing from one or more of the others. One or more of the CE, PMC(s), and/or the CE can be independent from and separately operable from either or both of the others, each of the PMC(s), UIC, and CE including different electronic processors capable of parallel, concurrent, and/or independent operation. In some embodiments, any, some, or all of the UIC, CE, and PMC(s) are capable of operational isolation from any, some, or all of the others such that if one or more of the others turn off, stop working, encounter an error or enter a failure mode, reboot, and/or reset, then the operationally isolated one(s) can continue functioning without interruption of user service, without turning off, without being operationally affected, and/or without being detrimentally operationally affected. The UIC, PMC(s), and/or CE can be operationally isolated, and/or physically or functionally separated, and still be configured within the housing 20 of the pump 10 to be in electronic communication with each other, transmitting data and/or instructions between or among each of them as needed. While functioning properly in the normal course of operations, any, some, or all of the UIC, CE, and PMC(s) can operate in simultaneous, real-time parallel and/or concurrent processing modes, such that any, some, or all of the UIC, CE, and PMC(s) are capable of performing different operations and tasks at generally the same time.

In some embodiments, there is frequent, periodic electronic communication between two or more of the UIC, CE, and PMC(s). For example, a “heartbeat” signal or other short, consistent, and/or repeating confirmatory signal can be sent between or among any two or more of the UIC, CE, and PMC(s) to enable each of the controllers in such communication to verify that the other controller(s) in such communication is or are still viable, powered on, and/or functioning. When one controller does not receive the confirmatory signal from another, it can be configured to stop functioning immediately or stop functioning after a short delay (e.g., at least about 10 seconds or at least about 30 seconds), enter into a limited functionality mode, and/or display an error message or provide another advisory communication. Any of the UIC, PMC(s), and/or CE can be considered capable of separate, isolated, and/or independent functionality if it can continue on with its operations normally, or substantially or entirely unaffected, after failing to receive a confirmatory signal from another controller, or after another controller malfunctions, turns off, and/or stops communicating.

In some arrangements, two or more controllers are capable of separate, isolated, and/or independent functionality for a limited amount of time. For example, in some embodiments the UIC and PMC can be configured to communicate a confirmatory signal between them. If one of the UIC or PMC does not receive the confirmatory signal from the other, then it can continue on its normal operations without any significant interruption or modification for a safety period, such as a period of time that is sufficiently long for the temporarily silent controller to stop and return to normal operations (e.g., to reboot, to reset, to enter and exit a failure mode, to power off and back on, to receive an update, and/or to identify and correct an internal operating error, etc.) but no so long as to present a significant risk of harm to a patient caused by independent operation. In some embodiments, the safety period can be at least about 30 seconds or at least about 1 minute, but no more than about 3 minutes, no more than about 5 minutes, or no more than about 10 minutes, or some other appropriate amount of time under particular operating circumstances. If the temporarily silent controller once again begins communicating the confirmatory signal before the expiration of the safety period, then the other controller that has been operating in a separate, isolated, and/or independent manner can continue on with normal operations; however, if the temporarily silent controller does not begin communicating the confirmatory signal before the expiration of the safety period, then the other controller can be configured to halt or modify its operations and/or communicate a warning or other advisory message.

The safety period can be different between different controllers. For example, in some embodiments, the safety period between the UIC and PMC may be shorter than the safety period between the UIC and CE and/or between the PMC and CE. In some embodiments, the safety period between the UIC and CE and/or between the PMC and CE can be no more than about half an hour or no more than about an hour or no more than about a day. In some embodiments, there may be no safety period, permitting indefinite separate, isolated, and/or independent operation between the UIC and CE and/or between the PMC and CE. In some embodiments, a shorter safety period for separate, isolated, and/or independent operation between the UIC and PMC may be needed to avoid operation of the pump driver 14 without the benefit of input from a user, without the benefit of one or more reports or alarms to the user from the UIC while the pump driver 14 continues pumping, and/or without the benefit of tracking of pumping by the UIC.

In some embodiments, any or all information, instructions, and/or programming entered by a user in the UIC can be automatically evaluated, checked, and/or validated for effectiveness and safety for the patient by one or more algorithms and/or data collections in the UIC before such information, instructions, and/or programming is conveyed or communicated, in whole or in part, and/or before any signal or instruction is sent, by the UIC to the PMC and/or the CE. The user is not typically permitted to modify, remove, and/or change system architecture or operating systems in normal operation of the pump 10.

FIG. 2 shows an example of a disposable fluid holder, such as a disposable cassette 50, that includes a plastic housing and a flexible, elastomeric silicon membrane. Any structure, material, function, method, or step that is described and/or illustrated in U.S. Pat. No. 4,842,584, which is incorporated herein by reference in its entirety, including but not limited to the pumping cassette, can be used by itself or with or instead of any structure, material, function, method, or step that is described and/or illustrated in this specification. The plastic housing of the cassette 50 can include one or more (e.g., two as shown) fluid inlets 52 and a fluid outlet 54 formed in a main body 56. The cassette 50 can be temporarily positioned for example in the loader 20 of a pump driver 14. The one or more fluid inlets 52 are coupled with one or more inlet tubes 57 in fluid communication with one or more sources of medical fluid, such as one or more IV bags, vials, and/or syringes, etc., containing medical fluid. If multiple inlets 52 and inlet tubes 57 are provided, as shown, then multiple sources of medical fluid can be simultaneously supplied to a patient through the cassette 50. The fluid outlet 54 is coupled to an outlet tube 55 in fluid communication with the patient, normally by way of a needle leading into a patient's blood vessel.

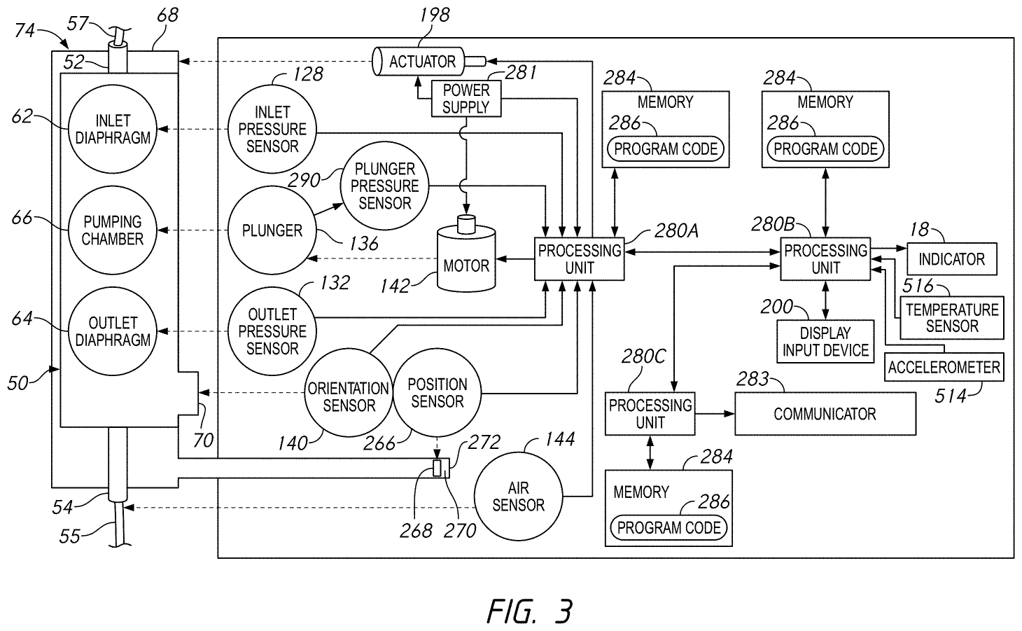

A flexible, elastomeric membrane forms a diaphragm 60 within a pumping chamber 66 on an inner face 68 of the main body 56. In operation, fluid enters through one or more of the inlets 52 and is forced through the outlet 54 under pressure. One or more fluid channels within the main body 56 of the cassette 50 convey the fluid between the inlets 52 and the outlet 54 by way of the pumping chamber 66. Before use, the cassette is typically primed with fluid, usually saline solution. A volume of fluid is delivered to the outlet 54 when a plunger 136 of the pump 10 (see, e.g., FIG. 3) displaces the diaphragm to expel the fluid from the pumping chamber 66. During an intake stroke, the plunger 136 retracts from the diaphragm 60, and the fluid is then drawn in through the inlet 52 and into the pumping chamber 66. In a pumping stroke, the pump 10 displaces the diaphragm 60 of the pumping chamber 66 to force the fluid contained therein through the outlet 54. In some embodiments, the directional movement of flow can be facilitated by one or more directional valve(s) (e.g., at one or more of inlet 52 or outlet 54). The fluid can flow from the cassette 50 in a series of spaced-apart pulses rather than in a continuous flow. In some embodiments, the pump 10 can deliver fluid to a recipient (e.g., a patient) at a pre-set rate, in a pre-determined manner, and for a particular (e.g., pre-selected) time or total dosage. The cassette 50 can include an air trap 59 in communication with an air vent (not shown).

FIG. 3 is a schematic diagram of functional electronic components for a medical pump (e.g., the pump 10 of FIG. 1) that can be used in connection with the disposable cassette 50 (see FIG. 2) for delivering a fluid to a patient. One or more processors or processing units 280 can be included in pump 10 that can perform various operations, some examples of which are described in greater detail below. Any two or more, or all, of the processing units 280 can be in electronic communication with each other, but in some embodiments can operate for some period independently from each other. The processing unit(s) 280 and all other electrical components within the pump 10 can be powered by a power supply 281, such as one or more components of power source 90 of pump 10. In some embodiments, the processing unit 280a can be configured as a pump motor controller (PMC) to control the electric motor 142 being energized by the power supply 281. When energized, the electric motor 142 can cause the plunger 136 to reciprocate back and forth to periodically actuate, press inward, and/or down-stroke, causing plunger 136 to temporarily press on pumping chamber 66, driving fluid through cassette 50. The motor 142, plunger 136, sensors 128, 290, 132, 140, 266, 144 can be included in or as an integrated part of the pump driver 14 of the pump 10. In some embodiments, as shown, the pump 10 can be configured so that the inlet pressure sensor 128 engages the inlet diaphragm 62 of cassette 50, and the outlet pressure sensor 132 engages the outlet diaphragm 64 of cassette 50. When retracting, moving outward, or on an up-stroke, the plunger 136 can release pressure from pumping chamber 66 and thereby draw fluid from inlet 52 into pumping chamber 66. In some implementations of cassette 50, a flow stop 70 is formed as a pivotal switch in the main body 56 and protrudes a given height from the inner surface 68. This protrusion forms an irregular portion of the inner surface 68 which can be used in some embodiments to align the cassette 50 as well as monitor the orientation of the cassette 50. In some embodiments, one form of a flow stop 70 can provide a manual switch or valve for closing and opening the cassette 50 to fluid flow.

In some embodiments, the processing unit 280a can control a loader 20 of the pump 10 with an electronic actuator 198 and a front carriage 74 being energized by the power supply 281. When energized, the actuator 198 can drive the front carriage 74 between closed or open positions. The front carriage 74 in the open position can be configured to receive the cassette 50 and in the closed position can be configured to temporarily securely retain the cassette 50 until the front carriage 74 is moved to the open position. A position sensor 266 for the cassette 50 can be provided in the pump 10. The position sensor 266 can monitor the position of a slot 268 formed in a position plate 270. The position sensor 266 can monitor a position of an edge 272 of a position plate 270 within the pump 10. By monitoring the position of the position plate 270, the position sensor 266 can detect the overall position of the front carriage 74 of the loader 20. The position sensor 266 can be a linear pixel array sensor that continuously tracks the position of the slot 268. Of course, any other devices can be used for the position sensor 266, such as an opto-tachometer sensor.

A memory 284 can communicate with the processing unit 280a and can store program code 286 and data necessary or helpful for the processing unit 280 to receive, determine, calculate, and/or output the operating conditions of pump 10. The processing unit 280a retrieves the program code 286 from memory 284 and applies it to the data received from various sensors and devices of pump 10. The memory 284 and/or program code 286 can be included within or integrally attached to (e.g., on the same circuit board) as the processing unit 280a, which in some embodiments can be the configuration for any processor or processing unit 280 in this specification.

In some embodiments, the program code 286 can control the pump 10 and/or track a history of pump 10 operation details (which may be recorded and/or otherwise affected or modified, e.g., in part by input from sensors such as air sensor 144, position sensor 266, orientation sensor 140, outlet pressure sensor 132, plunger pressure sensor 290, inlet pressure sensor 128, etc.) and store and/or retrieve those details in the memory 284. The program code 286 can use any one or more of these sensors to help identify or diagnose pumping problems, such as air in a pumping line, a pumping obstruction, an empty fluid source, and/or calculate expected infusate arrival time in a patient. The display/input device 200 can receive information from a user regarding a patient, one or more drugs to be infused, and details about a course of infusion into a patient. The display/input device 200 can provide a clinician with any useful information regarding the pumping therapy, such as pumping parameters (e.g., VTBI, remaining volume, infusion rate, time for infusion, elapsed time of infusion, expected infusate arrival time, and/or time for completion of infusion, etc.) Some or all of the information displayed by the display/input device 200 can be based on the operation details and calculations performed by the program code 286.

In some embodiments, the operation details can include information determined by the processing unit 280a. The processing unit 280a can process the data from pump 10 to determine some or all of the following operating conditions: whether or when the cassette 50 has been inserted, whether or when the cassette 50 is correctly oriented, whether or when the cassette 50 is not fully seated to the fixed seat 162, whether or when the front carriage assembly 74 is in an open or closed position, whether or when a jam in the front carriage assembly 74 is detected, whether or when there is proper flow of fluid through the cassette 50 to the patient, and whether or when one or more air bubbles are included in the fluid entering, within, and/or leaving cassette 50. The processing unit 280a can be configured to determine one or more operating conditions to adjust the operation of the pump 10 to address or improve a detected condition.

For example, the processing unit 280a can receive data from a plunger pressure sensor 290 operatively associated with the plunger 136. The plunger pressure sensor 290 can sense the force on plunger 136 and generate a pressure signal based on this force. The plunger pressure sensor 290 can communicate with the processing unit 280a, sending the pressure signal to the processing unit 280a for use in helping to determine operating conditions of pump 10.

The processing unit 280a can receive an array of one or more items of pressure data sensed from the cassette inner surface 68 determined by the plunger pressure sensor 290 and inlet and outlet pressure sensors 128 and 132. The processing unit 280a can combine the pressure data from the plunger pressure sensor 290 with data from inlet and outlet pressure sensors 128 and 132 to provide a determination as to the correct or incorrect positioning of cassette 50. In normal operation, this array of pressure data falls within an expected range and the processing unit 280a can determine that proper cassette loading has occurred. When the cassette 50 is incorrectly oriented (e.g., backwards or upside down) or when the cassette 50 is not fully seated to the fixed seat 162, one or more parameters or data of the array of pressure data falls outside the expected range and the processing unit 280a determines that improper cassette loading has occurred.

As shown, in some embodiments, the processing unit 280a can receive data from one or more air sensors 144 in communication with outlet tube 55 attached to the cassette outlet 54. An air sensor 144 can be an ultrasonic sensor configured to measure or detect air or an amount of air in or adjacent to the outlet 54 or outlet tube 55. In normal operation, this air content data falls within an expected range, and the processing unit 280a can determine that proper fluid flow is in progress. When the air content data falls outside the expected range, the processing unit 280a can determine that improper air content is being delivered to the patient.

Processing unit 280a can continuously or periodically communicate with an independent and separate processor or processing unit 280b to communicate information to the user and/or to receive data from the user that may affect pumping conditions or parameters. For example, processing unit 280a can communicate by wire or wirelessly with processing unit 280b which can be configured as a user interface processor or controller (UIC) to control the output and input of display/input device 200, including by displaying an operating condition and/or activate indicator 18 to communicate with a user. In some embodiments, processing unit 280b can receive user input regarding pumping conditions or parameters, provide drug library and drug compatibility information, alert a user to a problem or a pumping condition, provide an alarm, provide a message to a user (e.g., instructing a user to check the line or attach more fluid), and/or receive and communication information that modifies or halts operation of the pump 10.

An independent and separate processor or processing unit 280c can be configured as a communications engine (CE) for the pump, a pump communications driver, a pump communications module, and/or a pump communications processor. Processing unit 280c can continuously or periodically communicate with processing units 280a and 280b to transmit and/or receive information to and from electronic sources or destinations separate from, outside of, and/or remote from, the pump 10. As shown, processing unit 280c can be in electronic communication with or include a memory 284 and program code 286, and processing unit 280c can be in communication with and control data flow to and from a communicator 283 which can be configured to communicate, wired or wirelessly, with another electronic entity that it separate from the pump 10, such as a separate or remote user, a server, a hospital electronic medical records system, a remote healthcare provider, a router, another pump, a mobile electronic device, a near field communication (NFC) device such as a radio-frequency identification (RFID) device, and/or a central computer controlling and/or monitoring multiple pumps 10, etc.

As shown schematically in FIG. 3, a pump 10 can be provided with many components to accomplish controlled pumping of medical fluid from one or more medical fluid sources to a patient. For example, one or more processors or processing units 280 can receive various data useful for the processing unit(s) 280 to calculate and output the operating conditions of pump 10.

The processing unit(s) 280 can retrieve the program code 286 from memory 284 and apply it to the data received from various sensors and devices of pump 10, and generate output(s). The output(s) are used to communicate to the user by the processing unit 280b, to activate and regulate the pump driver by the processing unit 280a, and to communicate with other electronic devices using processing unit 280c.

As illustrated, all components and circuitry can be located inside of the housing 12 of the pump 10. In some embodiments, one or more of the components and/or portions of the circuitry can be located outside of the housing 12 and/or in another housing or device.

In some embodiments, the communicator 283 or any other portion of the electronics of the pump 10 can be or can comprise one or more of a wire, a bus, a receiver, a connector port, a transmitter, a transceiver, a modem, a codec, an antenna, a buffer, a multiplexer, a network interface, a router, and/or a hub, etc. The communicator 283 can communicate with another electronic entity in any suitable manner, such as by wire, short-range wireless protocol (Wi-Fi, Bluetooth, ZigBee, etc.), fiber optic cable, cellular data, satellite transmission, and/or any other appropriate electronic medium.

The pump 10 can comprise one or more sensors for determining general operating conditions and/or general standing conditions and/or physical movement of the pump 10. For example, the pump 10 can comprise one or more accelerometers 514 and/or one or more temperature sensors 516. In some embodiments, one of the accelerometers 514 can be configured to detect a falling motion of the pump 10, such as when the pump 10 is dropped or when a pole stand tips over with the pump 10 attached, and another of the accelerometers 514 can be configured to detect the severity of an impact (e.g., “G-shock”) when the pump 10 hits the floor or another object during or after a fall. In some implementations, the same accelerometer 514 can be configured to detect both falling and impact severity. One or more of the accelerometers 514 can be in electronic communication with the UIC processor 280b as shown in FIG. 3. The processor 280b can be configured to receive one or more electrical signals from one or both of the accelerometers 514 that can enable the processor 280b to determine that a fall occurred and/or that can provide various data about a fall of the pump 10, such as the magnitude of the acceleration during the fall, the time duration of the fall, the occurrence of one of more impacts in the fall, and/or the force of impact(s) from the fall.

The processor 280b can be configured to record the date and time when the fall occurred and store this information, along with one or more items of data from the accelerometers regarding the fall, in a memory 284. The processor 280b can communicate any or all fall data to the CE processor 280c which can be configured to send an electronic notice or message to one or more other local or remote locations that a fall occurred and/or to provide information about the fall. The processor 280b can communicate any or all fall data or another electronic signal to the PMC processor 280a which can, depending on the severity of the fall, cause the processor 280a to stop pumping and/or to refrain from pumping again until the pump 10 is fully inspected and/or repaired due to any damaged caused by the fall. The processor 280b can be configured to provide a warning or notification on the display 200 and/or via a speaker, haptic vibrator, indicator light, and/or by any other user-communication system or component to alert a user that a fall has occurred and/or that the pump 10 will not be permitted to function unless and until an inspection or repair occurs. In some embodiments, the processor 280b can require that an override authorization indicator such as a code, key, specialized tool, and/or other device or communication be provided by an authorized inspector and/or repair technician before the pump 10 will operate after a fall.

In some embodiments, one or more accelerometers 514 can be used to determine when the pump 10 is moving (e.g., when the poll stand to which the pump is attached is moving on its wheels to a different location) and/or the orientation sensor 140 can be used to determine when the orientation of the pump 10 has changed in a significant way (e.g., when the pump 10 has been temporarily oriented face-down on a shelf or on a bed). Any movement, change in location, and/or change in orientation can indicate that the pump 10 may be in a situation of increased risk of inadvertent touch screen contact or performance. For example, when the pump 10 is moving, a person has typically grasped the pump 10 or poll stand and is pushing it along. If the person inadvertently grasps a portion of the touch screen while pushing, or if the touch screen of the pump 10 encounters another object as it moves (e.g., an IV line, a curtain, a hospital gown, etc.), then an unintended and potentially disruptive or dangerous input can be received by the touch screen. To diminish this risk, when a controller in the pump 10 (e.g., the UIC) receives a signal from one or more accelerometers indicating movement, change in location, or change in orientation of the pump 10, the controller can temporarily disable or lock the touch screen to prevent or limit user input until the movement or change in orientation halts or returns to a normal state or until the user performs an action (e.g., entering a code or touch sequence, or pushing a button, etc.) to unlock, restore, reset, or otherwise enable the normal functioning of the pump 10.

When the pump 10 is operating at full capacity, which may in some circumstances involve pumping fluid from either or both pumps, displaying information on a screen, charging the battery, and/or communicating information to or from a remote source, the aggregated heat output from all of its components can be significant. Also, if any component of the pump malfunctions in a way that increases its heat output, it can create a risk of damaging other components and/or degrading, altering, and/or halting the performance of the pump 10. Thus, monitoring and managing the temperature of the pump 10 can be advantageous.

The pump 10 can include one or more temperature sensors 516 that can comprise any suitable electronic component or components that is or are configured to measure and/or convey information about an air temperature inside or in the environment of the pump 10. For example, in some embodiments, a temperature-variable resistor or thermistor, a thermocouple, an infrared sensor, and/or a temperature-variable diode or transistor can be used to create an electrical signal indicative of temperature. The pump 10 can include one or more temperature sensors 516 within the housing 12 of the pump 10 or outside of the housing 12 of the pump. In some embodiments, a plurality of temperature sensors 516 can be provided within the housing 12 that are spaced apart from each other and/or are located strategically in one or more different places within the housing 12 that are vulnerable or prone to, or have a tendency toward, over-heating, such as proximate to the battery 94, the display 200, the pump motor(s) 242, and/or any other component or place where over-heating is prone to occur.

The temperature sensor(s) 516 can be in electronic communication with the UIC processor 280b as shown in FIG. 3. The processor 280b can be configured to determine when one or more temperature sensors 516 have detected that an acceptable operating temperature has been exceeded (e.g., equal to or greater than about 100 degrees Fahrenheit or 38 degrees Celsius). If the pump 10 includes multiple temperature sensors 516, the processor 280b can be configured to determine that one or more specified regions within the pump 10 have exceeded an acceptable operating temperature.

The processor 280b can automatically respond to an excess temperature indication from one or more temperature sensor(s) 516 in any suitable way, including: (a) providing a notice to the user on the display 200 that the pump 10 is overheating; (b) communicating to the processor 280c, which can communicate a notice outside of the pump, such as to a local or remote location, that the pump 10 is overheating; (c) storing in memory 284 information about an over-heating incident, including the duration and temperature range of the overheating period; (d) deactivating, turning-off, slowing down, modifying, and/or delaying, one or more or all functions or operations of the pump 10 and/or one or more or all functions or operations of the pump 10 that are considered to be non-critical and/or not time-sensitive, and/or that are considered to be significant causes of heat generation, such as electrical charging and/or sending or receiving communications outside of the pump 10 (e.g., updating software or data from an external source). In some embodiments, the brightness of the display 200 can automatically be diminished by the processor 280b if a temperature sensor 516 in the region of the display 200 detects a temperature in excess of a permissible level. The processor 280b can be configured to use a hierarchy of functionality in determining how to manage the modifications in response to an excess temperature detection, such as by modifying the least critical or highest power-consuming function(s) first and/or by modifying the function(s) performed in the region of a detected temperature increase first. When the processor 280b has performed a temperature-induced modification of functionality, and then later one or more of the temperature sensors 516 detects that the temperature has dropped to or below an acceptable level (e.g., less than or equal to about 100 degrees Fahrenheit or 38 degrees Celsius), any modified functionality in the pump 10 and/or in the region of the previously detected increase in temperature can be automatically stopped and the operation of the pump 10 can automatically return to normal.

In a pump 10 with multiple pump drivers 14, many advantages can be derived and/or many problems can be avoided or diminished from coordinating, correlating, cross-checking, inquiring about, notifying and/or warning the user about, and/or monitoring, the pumping and/or pump programming of two or more of the pump drivers 14 together. For example, in some embodiments, one or more processors of a single pump system within the same housing can be configured to automatically halt pumping or programming of one or more pump drivers and/or change one or more pumping parameters of one or more pump drivers, and/or communicate with a user through a single display 200 (e.g., by providing a warning notification to the user, requesting further information or confirmation from the user, etc.), when one or more of the following occurs: (a) a fluid to be programmed or executed for infusion by one of the pump drivers is incompatible with, may cause harm or risk to a patient when infused with, and/or may diminish the effectiveness of, a fluid to be programmed or executed for infusion by another of the pump drivers; (b) an infusion rate and/or volume and/or type of a fluid to be programmed or executed for infusion by one of the pump drivers implies, suggests, and/or requires one or more physiological characteristics about a patient (e.g., weight, sex, age, disease, injury, mental state, pulse rate, blood pressure, blood clotting ability, and/or any other physical condition) that would be inconsistent with an infusion rate and/or volume and/or type of a fluid to be programmed or executed for infusion by another of the pump drivers; and/or (c) an infusion of one type of fluid by one of the pump drivers requires an infusion of another type of accompanying fluid by another of the pump drivers, but one of these pump drivers has been stopped, not started, and/or not programmed or executed for infusion of the required accompanying fluid.

In some embodiments, the coordinating, correlating, cross-checking, and/or monitoring of the pumping of fluid by two or more pump drivers within the same pump system can help provide security, safety, protection, and/or effective therapy to a patient. For example, when a single processor or memory, and/or when a single pump system comprising multiple pump drivers contained within the same housing 12, is configured to track, monitor, and/or store information about, the pumping of two or more pump drivers, then a patient's total dosage(s) of all fluids within a particular pumping session or a particular course or treatment or a particular stay in a healthcare facility can be determined and stored in one memory and/or in one device or system, at the origin of the pumping. This approach can provide advantages over electronically sending or communicating pumping information about a particular patient from or between multiple, separate pump drivers in separate housings and/or outside of the same housing into a common electronic medical record system or into a remote processor where such information can be aggregated and/or added together. For example, in such a detached or separated system, one or more errors or inefficiencies can be created if the communication between the individual pump drivers and the electronic medical record system or remote processor is impeded, disconnected, inaccurate, or does not occur (such as in the case of a network or other electronic communication failure), or if there is a delay in the communicated information.

In some embodiments, when a first pump driver has completed a course of infusing a first fluid at a first infusion rate and then a second pump driver is set to begin in sequence a different course of infusing a second fluid at a different rate, the second pump driver can be configured to start at the rate of the previously concluded first pump driver for the first fluid so that the first fluid that is still located in the patient fluid line downstream from the pump cassette can continue to be infused at the first rate that is appropriate for such fluid until the first fluid passes out of the fluid line and into the patient, at which point the second pump driver can switch to the second infusion rate. The processor that is controlling the pumping of the second pump driver can determine how long to pump at the first rate based upon an estimate or determination of the volume of fluid between the cassette and the patient.

The one or more pump drivers 14 have many moving parts that can wear down, break, malfunction, and/or fall out of calibration over time after extended use. Either or both pump drivers 14 may require servicing or replacement more often than other components of the pump 10. When the pump driver 14 has a problem, it can quickly lead to inaccurate, unreliable, or failed pumping of fluid to the patient. In some embodiments, either or both of the pump drivers 14 can be provided as a discrete, modular, and/or a readily, conveniently, and/or quickly separable or removable, self-contained unit from other components in the pump 10 and/or from within the housing of the pump 10.

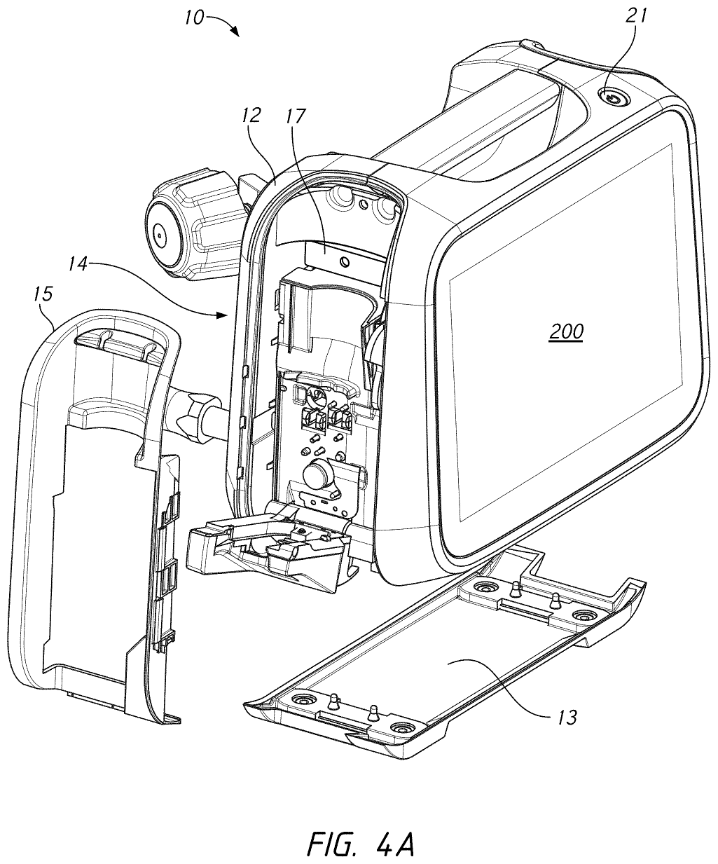

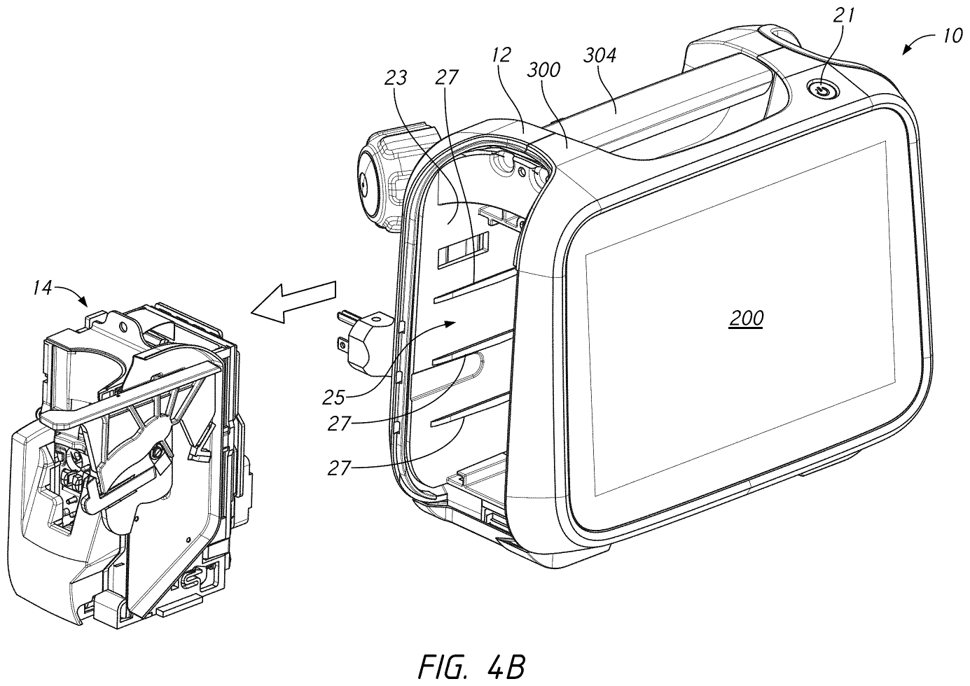

As shown in FIG. 4A-4B, either or both pump drivers 14 can be removed, serviced, and/or replaced without affecting, and/or without significantly affecting, other components of the pump 10. For example, a left pump driver 14 can be removed while a right pump driver 14 continues to be usable by a health care facility; or either or both pump drivers 14 can be removed while the UIC or CE continues to be able to receive programming instructions from a user and/or updates or information from a remote source.

FIG. 4A illustrates that in some embodiments a bottom cover 13 and/or a side cover 15 can be easily removed from a location near and/or generally surrounding a region of the pump 10 that includes the pump driver 14, such as by removing one or more connectors such as screws. These one or more covers 13, 15 can generally protect the pump driver 14 during use and storage from ingress of liquid, debris, and/or other unintended contact. After removal of the one or more covers 13, 15, at least a front side of the pump driver 14 is exposed but the pump driver 14 can remain attached to the overall pump 10. In some embodiments, as shown, the one or more covers 13, 15 extend across or over at least a portion of the front of the pump driver 14 and/or across or over at least a portion of a side of the pump driver 14, but the one or more covers 13, 15 do not extend past the back side of the pump driver 14. As shown, in some embodiments, removing the one or more covers 13, 15 does not remove either or both of the pump drivers 14 from the pump 10. One or more mounting brackets 17 or other couplings or connectors can removably affix, couple, and/or secure the pump driver 14 to and/or inside the pump 10. The one or more mounting brackets 17 and/or other couplings and/or connectors can be removed from the pump 10 to release the pump driver 14 from the pump 10, such as by removing one or more connectors such as screws. As shown in FIG. 4B, after the pump driver 14 is no longer secured to and/or inside of the pump 10, the pump driver 14 can be withdrawn as a modular unit from a pump driver cavity 23 in the pump 10. A portion of the housing 12 that generally surrounds and extends past the back of one of more of the pump drivers 14 during use of the pump 10 can be configured to remain in place while and after the pump driver(s) 14 are removed from the pump 10.

In some embodiments, as illustrated, the mounting bracket is configured to attach the pump driver(s) 14 to the inside of the pump 10, not to the portion of the housing 12 that generally surrounds and extends past the back of one or more of the pump drivers 14 during use of the pump 10. The removal of the pump driver 14 may require or permit easy and fast disconnection of one or more cables and/or electrical wires with quick-detach electrical couplings (not shown).

As shown, the pump driver cavity 23 can include an interior isolating wall or partition within the housing 12 that separates or seals the pump driver 14 from any, some, or all of the other components or parts of the pump 10. The isolating wall or partition can be useful in protecting one or more other components or parts of the pump 10 (e.g., the battery, the display, and/or any or all of the electronic components or memory or controllers) from damage, wear, and/or contamination caused by the pump driver 23 and/or cassette 50. For example, the pump driver 23 may produce undesirable vibration, heat, and/or potential contamination from human contact with the cassette 50 and/or leaking liquid from a potentially malfunctioning or improperly inserted or used cassette 50. When the pump driver 14 is removed, the pump driver cavity 23 can remain sealed off and/or separated from the interior of the pump 10 that contains any one or more other components of the pump 10 by the isolating wall or partition. In some embodiments, the pump 10 does not need to be requalified after servicing or replacing the pump driver 14 because the interior of the pump 10 with the controlling electronics or power source has not been opened or exposed.

In a process or method of repairing and/or servicing the pump 10, a worn, damaged, broken, and/or uncalibrated pump driver 14 can be removed, as shown in FIGS. 4A-4B, and a new and/or functioning pump driver 14 can quickly and easily be: (a) connected electrically using the electrical couplings, (b) inserted into the pump driver cavity 23 in the housing 12 of the pump 10, and/or (c) secured, coupled, and/or affixed to the pump 10 using one or more of the mounting brackets 17 or other couplings or connectors, essentially in the reverse order as shown in FIGS. 4A and 4B. In some embodiments, the pump driver 14 can be repaired, serviced, and/or calibrated locally, near the pump 10, or in the same health care facility where the pump 10 is used, and then returned and secured into the pump driver cavity 23 without replacing the pump driver 14.

As illustrated in FIGS. 4A and 4B, one or more modular pump drivers 14 can be attached to the pump 10 by a small number of brackets 17, couplings, or connectors, such as less than or equal to five, less than or equal to three, or two or less (as shown). Using only a small number of brackets 17, couplings, or connectors can permit the removal, servicing, and/or replacement of the pump driver 14 quickly and easily. One or more supporting regions 25 can be provided on the pump driver 14 and/or in the pump driver cavity 23 to help securely and removably attach the pump driver 14 to the pump 10 to help resist vibration, jostling, and/or other undesirable movement during use without requiring additional brackets 17, couplings, or connectors. For example, as shown, a supporting region 25 can comprise one or more ribs or rails 27 configured in size, shape, and/or orientation to tightly fit against and support the outside profile of the modular pump driver 14, helping a technician to rapidly align and/or secure the pump driver 14 in place while simultaneously permitting generally horizontal sliding movement and removal of the pump driver 14 from the cavity 23 when the one or more brackets 17, couplings, and/or connectors are removed.

One or more modular pump drivers 14 can be provided as shown with multiple parts and components secured directly or indirectly to each other in a module that is easily removable as a unit from the pump cavity 23, the housing, and/or another portion of the pump 10. In some embodiments, any, some, or all of the multiple parts and components of the module may not be individually directly secured to the pump cavity 23, to the housing 12, and/or to another portion of the pump 10.

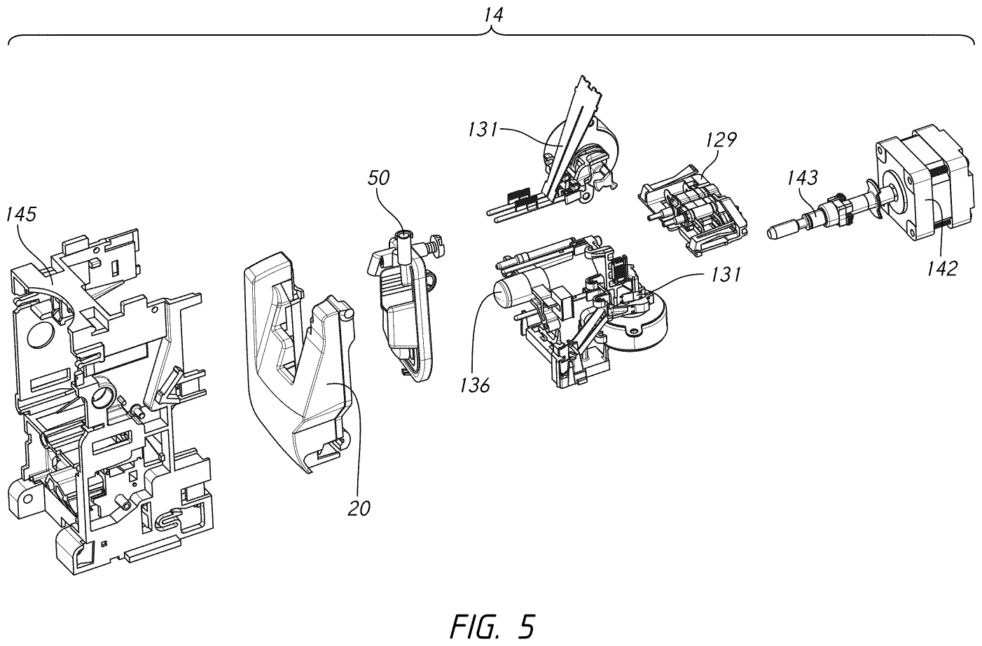

For example, as shown in FIG. 5, the modular pump driver 14 can include multiple parts and components in the form of a pump driver chassis 145, the pump motor 142 with a pumping actuator such as a plunger driving shaft 143 as shown, the cassette loader 20, the plunger 136, one or more sensors 129 (e.g., one or more pressure or position sensors), one or more valve actuators 131 configured to actuate valves in the cassette 50 when inserted into the cassette loader 20, and/or the PMC (not included as part of the illustrated pump driver 14 and not shown in FIG. 5). As shown, in some embodiments, the multiple parts and components of a modular pump driver 14 can each be indirectly secured to each other by way of attachment to one or more other components or parts of the module and/or by way of attachment or coupling to a common sub-housing, frame, chassis, substrate, and/or platform, etc., of the module. The pump driver 14 can be modular in that by disconnecting a small number of brackets 17, or other couplings or connectors from the pump cavity 23, the housing 12, and/or another portion of the pump 10, the entire module with each of the components and parts can be easily removed as a unit from the pump cavity 23, housing 12, and/or another portion of the pump 10 while the module remains substantially or completely intact and internally connected with its multiple parts.

As illustrated in FIG. 5, in some embodiments the modular pump driver 14 does not include an outer housing of its own. Rather, in this example, the chassis 145 can merely serve as a support to provide structure, rigidity, and/or positional stability for the other components and parts of the pump driver 14 during use, but the chassis 145 in this example does not serve as an exterior covering or protecting layer. As illustrated, the modular pump driver 14, when withdrawn from the pump driver cavity 23, is exposed and unprotected from ingress or liquid, debris, and undesirable contact. In the embodiment shown, there is no need for an outer housing because the modular pump driver 14 is contained and protected during use within the main housing 12 of the pump 10 with the other components of the pump 10, including the PMC(s), the UIC, the CE, the shared battery, the display/input device 200, and/or the communicator 283. In some embodiments, as shown, the modular pump driver 14 is not separated and/or cannot be separated from the housing 12 of the pump 10 while retaining or including a housing around or surrounding the exterior of the modular pump driver 14. It can be advantageous not to include a separate housing for the modular pump driver 14 because such a housing would otherwise create additional and unnecessary work for a technician who is repairing or servicing the modular pump driver 14 by requiring that the separate housing for the modular pump driver 14 be opened up and/or removed to access the components and parts of the pump driver 14. In this example, the modular pump driver 14 does not have its own battery or other power source or standard wall-socket power connector, and cannot function at all or cannot function safely and/or reliably when detached from the pump 10.

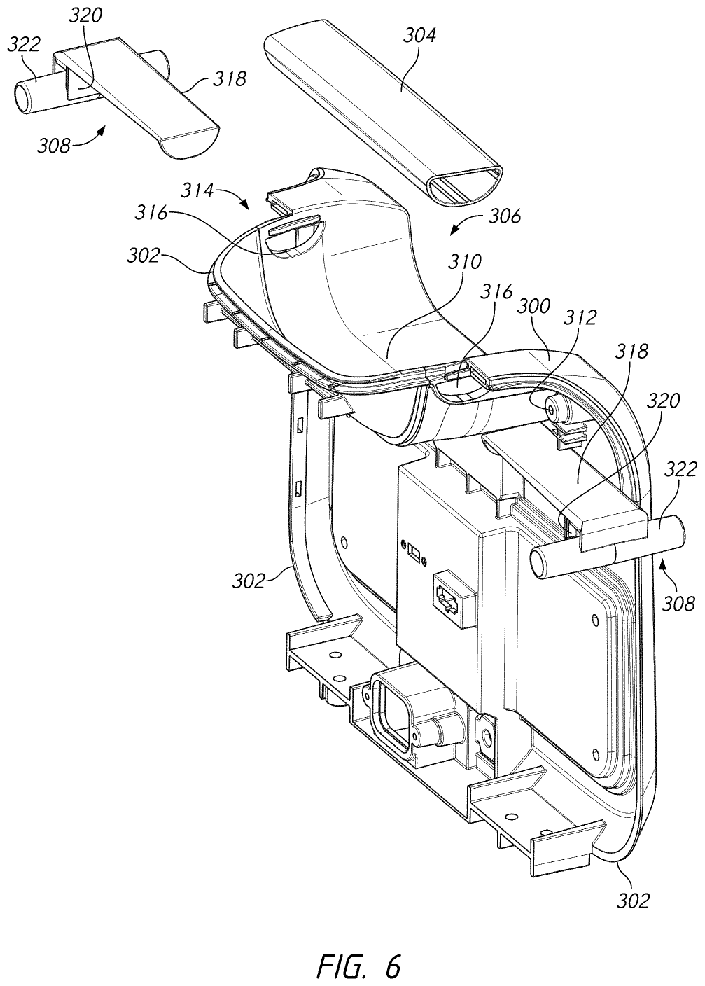

The housing 12 of the pump driver 14 can include a plurality or an assembly of discrete or separately manufactured panels, which can include a front panel 300 as shown in FIG. 6. Each panel, including panel 300, can include an edge 302 that closely, tightly, and/or securely abuts, connects with, interlocks, and/or attaches to a boundary region of another panel. A boundary region can be formed where a plurality of edges 302 of a plurality of panels meet, contact, and/or connect. In some embodiments, the boundary region can be configured to resist ingress of liquid, debris, contaminants, and/or any other undesirable substances. An elastomeric and/or resilient seal or gasket can be provided along one or more regions, or all or substantially all, of the boundary that can be compressed when a plurality of edges 302 come into proximity to each other. A handle 304 can be provided to assist a user in securely holding the pump 14 while moving the pump 14 and/or while securing the pump 14 in a stable location, such as on an IV pole stand. The handle 304 can comprise a portion of a handle assembly 306.

In some embodiments, the handle assembly 306 can include the handle 304, one or more handle connectors 308, a finger recess 310 formed in the panel 300, one or more handle seals (not shown), and/or one or more anchor regions 312. As illustrated, the handle 304 can be formed of a generally hollow, tubular member made of a generally rigid material such as plastic or metal that comprises a plurality of (e.g., first and second) end openings 314. The handle 304 can comprise a first surface, such as a lower surface, that is curved, rounded, ergonomic, and/or otherwise configured to comfortably fit against curled and/or grasping fingers. The handle 304 can comprise a second surface, such as an upper surface, that is generally flat and/or generally planar to securely fit and/or abut against the palm of a grasping hand. In some embodiments, when the handle 304 is secured to the housing 12, the second surface can be generally continuous with and/or generally collinear with a top or upper edge of the panel 300 and/or the housing 12 of the pump 10. As shown, the finger recess 310 can be a concave portion, cut-out, notch, and/or opening in the housing 12 that is sufficiently wide and deep to accommodate the insertion or presence of a user's fingers when a user grasps the handle 304 of the pump 10.

The finger recess 310 and/or any other suitable portion of the panel 300 can include a plurality of (e.g., first and second) attachment openings 316. During assembly or manufacturing of the handle assembly 306, the handle 304 can be brought into or near the finger recess 310 or other suitable portion of the panel 300 such that the first opening 314 of the handle 304 is immediately adjacent to the first attachment opening 316 and the second opening 314 of the handle 304 is immediately adjacent to the second attachment opening 316. With the handle 304 positioned in this location and orientation, a projection 318 on the first handle connector 308 can be inserted into and through the first attachment opening 316 and the first opening 314 of the handle 304, and a projection 318 on the second handle connector 308 can be inserted into and through the second attachment opening 316 and the second opening of the handle 304. Each of the respective projections 318 of the first and second handle connectors 308 can be advanced into the interior of the handle 304 until respective interior abutting surfaces 320 of the handle connectors abut a securing region generally surrounding each of the first and second attachment openings 316. The lengths of the respective projections 318 of the handle connectors 308 can be selected so that when the handle connectors 308 are fully inserted into the handle 304, the respective ends of the projections 318 of the handle connectors 308 are in close proximity with and/or abut each other. The respective handle connectors 308 can each be secured to the housing 12 in this position by affixing a connector, such as a screw, through a passage in a respective holder 322 in the respective handle connectors 308 to the respective anchor region 312 of the panel 300.

The one or more handle seals can be provided along and/or around the boundary, seam, and/or interface between the respective first and second end openings 314 of the handle 304 and the respective first and second attachment openings 316 of the finger recess 310. The seal(s) can be provided separately in the handle assembly 306 or the seal(s) can be attached to and/or molded with one or more portions of the handle assembly 306. For example, the seals can be provided at, near, and/or adjacent to the abutting surfaces 320 of each of the projections 318 of the handle connectors 308 such that the seals are automatically located at the appropriate location for sealing when the handle connectors 308 are fully inserted into the handle 304.

The handle 304, when attached to the pump 10 as part of the handle assembly 306, can provide a sturdy, break-resistant portion on a heavy pump 10 with a plurality of pump drivers 14. As shown, the handle connectors 308 provide a way of connecting the handle 304 to the housing 12 and can provide a high interior reinforcing shear strength for the handle 304. In the illustrated example, the shaft of the handle 304 itself is formed separately from the panel 300 of the housing 12 to which it attaches, and the shaft of the handle 304 itself is formed unitarily as a single piece of material between the first and second openings 314. The unitary construction of the handle 304 can help resist strain or breakage when the handle 304 is bearing the weight of the pump 10 while the pump is held by a user's hand. In some embodiments, as shown, there is no seam or boundary between two or more panels of the housing 12 that runs along or is part of the handle 304. As illustrated, the handle 304 can be securely attached to but separate from and not part of a panel of the housing 12.

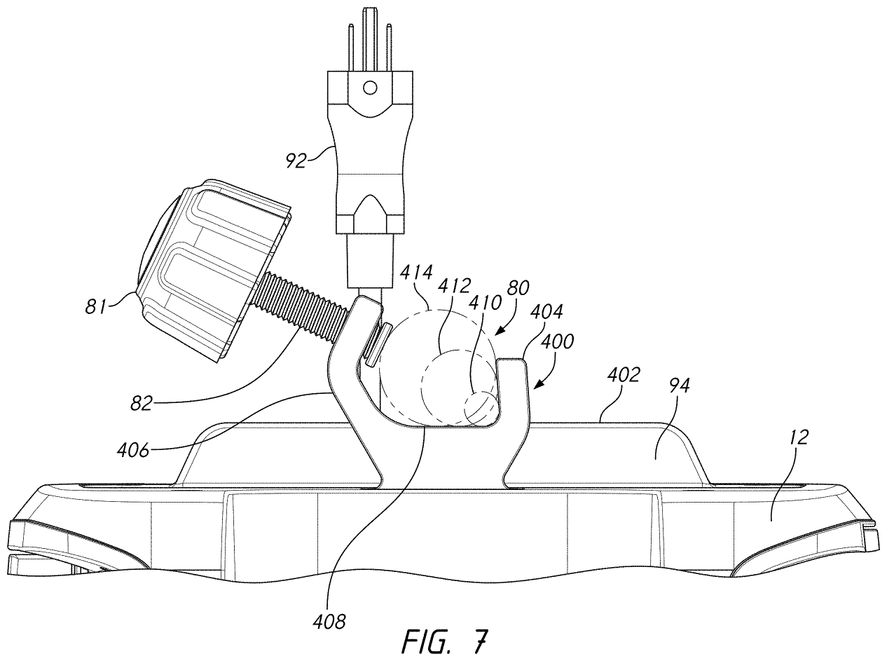

As shown in FIG. 7, a connector 80 can be affixed to the pump 10, such as on or through the housing 12. The connector 80 can be attached to the pump 10 in such a way that the weight of the pump 10 can be securely supported by the connector 80 when the pump 10 is suspended above the ground or in the air. For example, the connector 80 can be bolted onto the housing 12 and/or within the housing 12 of the pump 10. In some embodiments, as illustrated, the connector 80 can comprise an open and a closed position. In the open position, a movable actuator (such as a threaded shaft 82 with a rotatable knob 81) can be positioned (such as by rotation) to provide a void on and/or within the connector 80 that is configured to receive a pump support (such as an IV pole). For example, as shown, the actuator can be rotated via the knob 81 such that the shaft 82 is moved outward or away from the void. In the illustrated embodiment, a plurality of different IV poles 410, 412, 414 of different cross-sectional widths are shown (e.g., small, medium, and large). The connector 80 can be configured to receive and securely hold, with different adjustments, any one of the plurality of IV poles 410, 412, 414 without tipping or tilting the pump 10 during use, and/or while maintaining a substantially or virtually entirely vertically oriented position for the pump 10 that is substantially or virtually entirely parallel with the received and securely held IV pole 410, 412, 414 during use.

In some embodiments, as illustrated, the connector 80 can include a base 400 with a first lateral support 406, a second lateral support 404, and a central support 408. The actuator can be positioned, oriented, and/or otherwise configured to be supported by and/or be movable with respect to the first lateral support 406. For example, as shown, the threaded shaft 82 can be configured to selectively rotate inwardly and outwardly through a corresponding threaded hole (not shown) in the first lateral support 406. In the open position of the connector 80 (e.g., when the threaded shaft 82 is rotated in the outward direction or fully outwardly), a void can be formed between the lateral supports 406, 404 and the central support 408 that is sufficiently wide to receive any one of the plurality of IV poles 410, 412, 414 of different cross-sectional widths. The central support 408 can include a substantially or entirely flat surface that is essentially or entirely coplanar with a substantially or entirely flat portion of the rear surface 402 of the pump 10 that is adjacent to or at the same rearward position as a supporting region (such as a generally vertical linear region or a planar region) that is configured to touch, contact, align along, receive, and/or otherwise support the pump 10 against, the IV poles 410, 412, 414. As shown, in some embodiments, the supporting region can be the most rearward region of the pump 10 and/or of the housing 12 of the pump 10. In the illustrated example, the supporting region is located on the rear surface of the battery 94.

The electrical cable 92 can be positioned laterally away from the connector 80 and/or not along a vertical strip of the rear surface 402 of the pump 10 directly above and below the connector 80 and/or the central support 408, so that when the connector 80 receives one of the IV poles 410, 412, 414, the electrical cable 92 does not impede, resist, interfere with, and/or prevent any of the IV poles from being positioned flat and flush along the rear surface 402 of the pump 10.

In use, after one of a plurality of poles 410, 412, 414 is inserted into the void of the connector 80 in the open position, the actuator can be configured to advance into the void (e.g., by rotation of the knob 81) to exert a lateral force against an outer surface of the inserted pole 410, 412, or 414, as shown. The exerted force can cause the pole 410, 412, or 414 to move laterally against and/or exert a lateral force against the second lateral support 404. The second lateral support 404 can include a generally flat, generally planar surface that is generally perpendicular to a generally flat, generally planar surface of the central support 408. In some embodiments, as shown, the longitudinal axis of the actuator can be angled with respect to, or non-parallel with, either or both of the generally flat, generally planar surface of the central support 408 and/or a generally flat, generally planar surface of the second lateral support 404, such that as the actuator advances inward, the pole 410, 412, or 414 is moved along and/or is configured to exert a force simultaneously against both the second lateral support 404 and the central support 408.

As shown, the first lateral support 406 can include a first straight portion and a second straight portion, the first and second straight portions forming an angle between them and/or not being collinear with each other. The second lateral support 404 can be formed as a single straight portion. The first portion of the first lateral support 406 can be non-perpendicular with a surface on the central support 408. An inner surface of the second lateral support 404 can be generally or substantially perpendicular with the central support 408. The connector 80 can include a narrow neck with a cross-sectional width that is less than the widest distance between the inside surfaces of the first and second lateral supports 404, 406.

In a secured configuration, after fully moving the actuator into a holding position, the same connector 80 can be configured by adjustment to securely hold onto any one of a plurality of at least two different-sized poles 410, 412, or 414 with different cross-sectional widths in a secured coupling in which the connector 80 contacts each pole 410, 412, or 414 simultaneously at or along two or more or all of the following: (a) the actuator attached to the first lateral support 406; (b) the second lateral support 404; (c) the central support 408; and/or (d) a flat region on the rear surface 402 of the pump 10 that is substantially coplanar with, at substantially the same rearward position as, and/or adjacent to a flat region of the central support 408.

The connector 80 can be configured to hold the pump 10 generally or substantially vertically without any appreciable tipping or tilting. The pump 10 can be configured to attach to a pole 410, 412, 414 such that the pole 410, 412, 414 contacts the pump on at least two vertically separated points or regions on the rear of the pump 10 that form a substantially straight, substantially vertical line. For example, one of such points can be on the central support 408 of the connector 80 and the other of such points can be on the rear surface 402 of the housing 12, directly above or directly below the connector 80.

### TERMINOLOGY AND CONCLUSION

Reference throughout this specification to “some embodiments” or “an embodiment” means that a particular feature, structure, or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics may be combined in any suitable manner, as would be apparent to one of ordinary skill in the art from this disclosure, in one or more embodiments.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in this description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single disclosed embodiment.

Embodiments of the disclosed systems and methods may be used and/or implemented with local and/or remote devices, components, and/or modules. The term “remote” may include devices, components, and/or modules not stored locally, for example, not accessible via a local bus. Thus, a remote device may include a device which is physically located in the same room and connected via a device such as a switch or a local area network. In other situations, a remote device may also be located in a separate geographic area, such as, for example, in a different location, building, city, country, and so forth.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may be comprised of connected logic units, such as gates and flip-flops, and/or may be comprised of programmable units, such as programmable gate arrays, application specific integrated circuits, and/or processors. The modules described herein are preferably implemented as software modules, but may be represented in hardware and/or firmware. Moreover, although in some embodiments a module may be separately compiled, in other embodiments a module may represent a subset of instructions of a separately compiled program, and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with patients, health care practitioners, administrators, other systems, components, programs, and so forth.

A number of applications, publications, and external documents may be incorporated by reference herein. Any conflict or contradiction between a statement in the body text of this specification and a statement in any of the incorporated documents is to be resolved in favor of the statement in the body text.

Terms of equality and inequality (e.g., equal to, less than, greater than) are used herein as commonly used in the field, e.g., accounting for uncertainties present in measurement and control systems. Thus, such terms can be read as approximately equal, approximate less than, and/or approximately greater than. In other aspects of the invention, an acceptable threshold of deviation or hysteresis can be established by the pump manufacturer, the editor of the drug library, or the user of a pump.

While the embodiments of the invention disclosed herein are presently considered to be preferred, various changes and modifications can be made without departing from the scope of the invention. Although described in the illustrative context of certain preferred embodiments and examples, it will be understood by those skilled in the art that the disclosure extends beyond the specifically described embodiments to other alternative embodiments and/or uses and obvious modifications and equivalents. Thus, it is intended that the scope of the claims which follow should not be limited by the particular embodiments described above. The scope of the invention is indicated in the appended claims, and all changes that come within the meaning and range of equivalents are intended to be embraced therein.

The following is claimed:

1. A medical infusion pump system comprising:
a pump housing;
an electromechanical pump driver positioned within the housing, the pump driver configured to receive a disposable fluid holder and to pump medical fluid through the fluid holder;
an electronic pump motor controller positioned within the housing, the pump motor controller being in electrical communication with the pump driver; and
one or more additional electronic controllers within the housing, the one or more additional electronic controllers comprising one or more of a user interface controller in electrical communication with a user interface, or a communications engine in electrical communication with a communicator;
wherein the electronic pump motor controller is operationally isolated from the one or more additional controllers in that the electronic pump motor controller and the one or more additional controllers are each configured to be capable of continuing operation during at least a safety period when the other has stopped working, entered a failure mode, rebooted, or reset.

2. The pump system of claim 1, wherein the pump motor controller and the one or more additional electronic controller are located on separate printed circuit boards in different places within the pump housing.

3. The pump system of claim 1, wherein the one or more additional controllers comprises a user interface controller and a separate communications engine.

4. The pump system of claim 1, wherein each of the controllers is configured to communicate data or instructions with each of the other controllers.

5. A medical infusion pump system comprising:
a pump housing;
a user display;
a modular electromechanical pump driver positioned within the pump housing, the modular electromechanical pump driver comprising a first component in the form of a motor, a second component in the form of a loader configured to receive a disposable fluid holder, and a third component in the form of a plunger configured to pump medical fluid through the fluid holder;
wherein the first, second, and third components of the modular electromechanical pump driver are directly or indirectly connected to each other as a removable unit, and wherein the modular electromechanical pump driver can be removed as a unit from within the pump housing without removing the first, second, and third components from each other and without removing a portion of the pump housing that generally surrounds and extends past a back surface of the pump driver.

6. The pump system of claim 5, further comprising at least one more modular electromechanical pump driver.

7. The pump system of claim 5, further comprising a UIC processor or a CE processor.

8. The pump system of claim 7, wherein the UIC processor or the CE processor are capable of functioning when the modular electromechanical pump driver is separated from the pump.

9. A medical infusion pump system comprising:
a pump housing formed of a plurality of discrete panels with one or more boundaries between the panels of the pump housing;
a user display;
an electromechanical pump driver positioned on or within the pump housing;
a battery positioned on or within the pump housing; and
a handle connected to the pump housing, the handle formed as a unitary structure and the handle not including any portion of the boundary between panels of the pump housing.

10. The medical infusion pump system of claim 9, wherein the handle has a hollow interior from a first end to a second end, and the handle is connected to the pump housing through an internal connector inserted into the hollow interior of the handle.

11. The medical infusion pump system of claim 10, wherein the internal connector helps to reinforce the handle against breakage when the pump is held by a user.

12. The medical infusion pump system of claim 9, wherein an upper surface of the handle is continuous with an upper surface of the pump housing.

13. The medical infusion pump system of claim 12, wherein the upper surface of the handle and the upper surface of the pump housing is generally linear.

14. The medical infusion pump system of claim 9, wherein an upper surface of the handle is generally flat and a lower surface of the handle is generally curved.

15. The medical infusion pump system of claim 9, wherein the housing comprises a finger recess located near the handle.

16. The medical infusion pump system of claim 9, further comprising one or more seals located between the handle and the pump housing.

17. A medical infusion pump system comprising:
a pump housing;
a plurality of electromechanical pump drivers at least partially contained within the pump housing;
a battery positioned on or within the pump housing; and
one or more electronic processors within the pump housing in electrical communication with the plurality of pump drivers; and
a memory within the pump housing that is associated with the one or more electronic processors, the memory configured to contain information about one or more pumping parameters executed by or to be programmed for execution by at least two of the pump drivers.

18. The medical infusion pump system of claim 17, wherein the processor is configured to warn a user of the pump system if incompatible medical fluids are programmed to be infused through two different pump drivers.

19. The medical infusion pump system of claim 17, wherein the processor is configured to prevent the infusion of incompatible medical fluids through two different pump drivers.

20. A medical infusion pump system comprising:
a pump housing;
a display;
a processor;
at least one electromechanical pump driver at least partially contained within the pump housing;
a power source on or within the pump housing; and
a connector configured to separately receive and secure each one of a plurality of different-sized IV pole stands, the connector comprising a first surface that is perpendicular to a second surface, each of the first surface and the second surface being configured when the connector receives and secures each one of the plurality of different-sized IV pole stands to simultaneously contact said pole stand.

21. A combination of the pump system of claim 20 and one or more of the pole stands.

22. The pump system of claim 20, wherein the connector comprises an actuator.

23. The pump system of claim 22, wherein the actuator comprises a knob and a threaded shaft.

24. The pump system of claim 20, wherein the connector is configured to secure the pump system to each one of the plurality of pole stands without tilting.

25. A medical infusion pump system comprising:
a pump housing;
a display;
at least one processor;
at least one electromechanical pump driver at least partially contained within the pump housing;
a power source on or within the pump housing; and
one or more accelerometers configured to produce one or more electronic signals that indicates at least one of whether the pump system has fallen, is moving, or has changed orientation.

26. The medical infusion pump system of claim 25, wherein the one or more accelerometers is configured to determine impact severity from a fall.

27. The medical infusion pump system of claim 25, wherein the at least one processor is configured to lock the display against user input after receiving a signal from the one or more accelerometers.

28. The medical infusion pump system of claim 26, wherein the pump system is configured to store information that includes the day and time of a fall.

29. The medical infusion pump system of claim 25, wherein the at least one processor is configured to halt pumping after a fall until the pump system is inspected or repaired.

30. The medical infusion pump system of claim 25, wherein the at least one processor is configured to provide a message on the display indicating that a fall has occurred or that the pump system will not be permitted to function until after an inspection or repair has occurred.

31. A medical infusion pump system comprising:
a pump housing;
a display;
at least one processor;
at least one electromechanical pump driver at least partially contained within the pump housing;
a power source on or within the pump housing; and
one or more temperature sensors in electronic communication with the processor;
wherein the processor is configured to modify the performance of one or more operations of the pump system in response to an increase in temperature above an acceptable operating temperature.

32. The medical infusion pump system of claim 31, wherein the processor is configured to halt an updating process when the temperature sensor has detected an increase in temperature above the acceptable operating temperature.

33. The medical infusion pump system of claim 31, wherein the processor is configured to dim the display when the temperature sensor has detected an increase in temperature above the acceptable operating temperature.

34. The medical infusion pump system of claim 31, wherein the processor is configured to halt charging a battery when the temperature sensor has detected an increase in temperature above the acceptable operating temperature.

35. The medical infusion pump system of claim 31, wherein the processor is configured to delay or slow down one or more functions of the pump system in response to an increase in temperature above the acceptable operating temperature

36. The medical infusion pump system of claim 32, wherein the processor is configured to delay or slow down one or more functions of the pump system unless or until the temperature returns to the acceptable operating temperature.
